(19) United States
(12) Patent Application Publication
(10) Pub. No.: US 2023/0245766 A1
(43) Pub. Date: Aug. 3, 2023

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Haruyasu NAKATSUGAWA, Kanagawa (JP); Yasuhisa KANEKO, Kanagawa (JP); Kenji NAGAMIYA, Kanagawa (JP); Tomohide HIRAGAMI, Kanagawa (JP); Yasuyuki HOSONO, Kanagawa (JP); Nobuya KITAMURA, Kanagawa (JP); Keiji TSUBOTA, Kanagawa (JP)

(21) Appl. No.: 18/299,018

(22) Filed: Apr. 11, 2023

Related U.S. Application Data
(63) Continuation of application No. PCT/JP2021/035992, filed on Sep. 29, 2021.

(30) Foreign Application Priority Data
Oct. 28, 2020 (JP) 2020-180853

(51) Int. Cl. G16H 40/20; G06V 20/52

(52) U.S. Cl. CPC G16H 40/20; G06V 20/52

(57) ABSTRACT

An information processing apparatus, before at least one of a user who operates a control device controlling an imaging device that captures a medical image or a subject to be imaged by the imaging device is changed, notifies of a cleaning request of the control device based on information acquirable in a noncontact manner with the control device.

### CROSS-REFERENCE TO RELATED APPLICATIONS

This application is a continuation of International Application No. PCT/JP2021/035992, filed on Sep. 29, 2021, which claims priority from Japanese Patent Application No. 2020-180853, filed on Oct. 28, 2020. The entire disclosure of each of the above applications is incorporated herein by reference.

### BACKGROUND

### 1. Technical Field

The present disclosure relates to an information processing apparatus, an information processing method, and an information processing program.

### 2. Description of the Related Art

JP2010-061654A discloses that, in a case in which a tablet type information terminal device is used by a plurality of users, each time a change in the user is detected, the device outputs a message of transition to a cleaning mode and performs the transition to the cleaning mode.

JP2015-126985A discloses a stationary computer and a tablet type computer as a control device that controls an imaging device that captures a medical image.

### SUMMARY

In a technology described in JP2010-061654A, the change of the user is detected by logging in to the information terminal device. Therefore, the change of the user is detected by contacting the information terminal device by the second user, after the first user uses the information terminal device. Therefore, infectious disease may be transmitted from the first user to the second user through the information terminal device. In addition, a technology described in JP2015-126985A does not consider suppression of the spread of the infectious disease in the first place.

The present disclosure has been made in view of the above circumstances, and an object of the present disclosure is to provide an information processing apparatus, an information processing method, and an information processing program capable of suppressing the spread of the infectious disease through a control device controlling an imaging device that captures a medical image.

An information processing apparatus according to an aspect of the present disclosure is an information processing apparatus comprising at least one processor, in which the processor is configured to, before at least one of a user who operates a control device controlling an imaging device that captures a medical image or a subject to be imaged by the imaging device is changed, notify of a cleaning request of the control device based on information acquirable in a noncontact manner with the control device.

In the information processing apparatus according to the aspect of the present disclosure, the information is an imaging order including identification information of the user, and the processor may determine whether or not the user is changed by using the imaging order, and may notify of the cleaning request in a case in which it is determined that the user is changed.

In addition, in the information processing apparatus according to the aspect of the present disclosure, the processor may determine whether or not the user is changed by using identification information of the user obtained by short-range wireless communication with the control device, and may notify of the cleaning request in a case in which it is determined that the user is changed.

In addition, in the information processing apparatus according to the aspect of the present disclosure, the processor may notify of the cleaning request in a case in which it is detected that a second user different from a first user has entered an imaging room in which the control device is installed after the first user has exited the imaging room by using entry/exit information of the user with respect to the imaging room.

In addition, in the information processing apparatus according to the aspect of the present disclosure, the processor may determine whether or not the user is changed by using image information of the user imaged by a camera, who approaches the control device within a certain distance, and may notify of the cleaning request in a case in which it is determined that the user is changed.

In addition, in the information processing apparatus according to the aspect of the present disclosure, the processor may notify of the cleaning request by considering that the subject is changed in a case in which it is detected that imaging of the subject with the imaging device has been ended.

In addition, in the information processing apparatus according to the aspect of the present disclosure, the processor may detect that the imaging of the subject has been ended by using at least one of status information of the imaging device or status information of the control device.

In addition, in the information processing apparatus according to the aspect of the present disclosure, the processor may further notify of the cleaning request of the imaging device paired with the control device.

In addition, in the information processing apparatus according to the aspect of the present disclosure, the processor may notify of a warning in a case in which cleaning of the control device has not been performed even after a certain period of time has elapsed after the notification of the cleaning request.

In addition, in the information processing apparatus according to the aspect of the present disclosure, the processor may notify of a warning in a case in which a period of time from the notification of the cleaning request to a next use of the control device for which cleaning has not been performed is equal to or less than a predetermined threshold value.

In addition, in the information processing apparatus according to the aspect of the present disclosure, the processor may further perform control to prohibit use of the control device which is a target to be notified of the warning.

In addition, an information processing method according to another aspect of the present disclosure, that is executed by a processor included in an information processing apparatus, the method comprises notifying, before at least one of a user who operates a control device controlling an imaging device that captures a medical image or a subject to be imaged by the imaging device is changed, of a cleaning request of the control device based on acquirable information in a noncontact manner with the control device.

In addition, an information processing program according to still another aspect of the present disclosure, that causes a processor included in an information processing apparatus to execute a process comprises notifying, before at least one of a user who operates a control device controlling an imaging device that captures a medical image or a subject to be imaged by the imaging device is changed, of a cleaning request of the control device based on acquirable information in a noncontact manner with the control device.

According to the present disclosure, it is possible to suppress the spread of the infectious disease through a control device controlling an imaging device that captures a medical image.

### BRIEF DESCRIPTION OF THE DRAWINGS

FIG. 1 is a diagram showing a schematic configuration of an information processing system according to a first embodiment.

FIG. 2 is a block diagram showing an example of hardware configuration of a console and an information processing apparatus.

FIG. 3 is a block diagram showing an example of a functional configuration of the console.

FIG. 4 is a flowchart showing an example of cleaning request notification processing according to the first embodiment.

FIG. 5 is a diagram for explaining a first example.

FIG. 6 is a diagram for explaining a second example.

FIG. 7 is a diagram for explaining a third example.

FIG. 8 is a diagram showing a schematic configuration of an information processing system according to a second embodiment.

FIG. 9 is a block diagram showing an example of a functional configuration of the information processing apparatus.

FIG. 10 is a flowchart showing an example of cleaning request notification processing according to the second embodiment.

### DETAILED DESCRIPTION

Hereinafter, examples of an embodiment for implementing the technology of the present disclosure will be described in detail referring to the drawings.

### First Embodiment

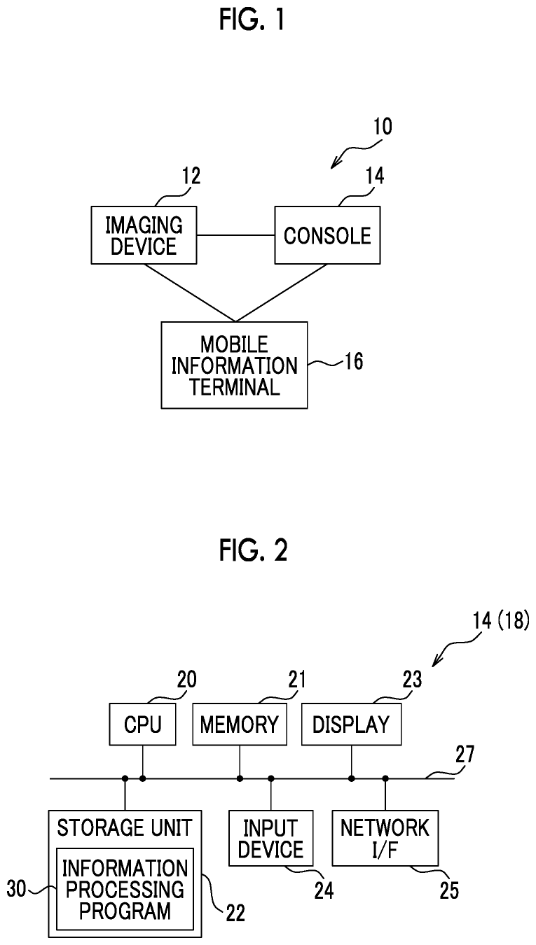

First, a configuration of an information processing system 10 according to the present embodiment will be described with reference to FIG. 1. As shown in FIG. 1, the information processing system 10 includes an imaging device 12, a console 14, and a mobile information terminal 16. The imaging device 12, the console 14, and the mobile information terminal 16 are communicably connected to each other. The mobile information terminal 16 is a shared terminal device. The console 14 is an example of an information processing apparatus according to the disclosed technology.

The imaging device 12 captures a medical image. Examples of the imaging device 12 include a computed tomography (CT) apparatus, a magnetic resonance imaging (MRI) apparatus, and a digital radiography (DR) cassette.

The mobile information terminal 16 is an example of a control device that controls the imaging device 12 together with the console 14. Examples of the mobile information terminal 16 include a tablet type computer, a smartphone, and the like.

Next, a hardware configuration of the console 14 according to the present embodiment will be described with reference to FIG. 2. As shown in FIG. 2, the console 14 includes a central processing unit (CPU) 20, a memory 21 as a temporary storage area, and a non-volatile storage unit 22. In addition, the console 14 includes a display 23, such as a liquid crystal display, an input device 24, such as a keyboard and a mouse, and a network interface (I/F) 25 that is connected to a network. The CPU 20, the memory 21, the storage unit 22, the display 23, the input device 24, and the network I/F 25 are connected to a bus 27.

The storage unit 22 is realized by a hard disk drive (HDD), a solid state drive (SSD), a flash memory, or the like. An information processing program 30 is stored in the storage unit 22 as a storage medium. The CPU 20 reads out the information processing program 30 from the storage unit 22, loads the information processing program 30 in the memory 21, and executes the loaded information processing program 30.

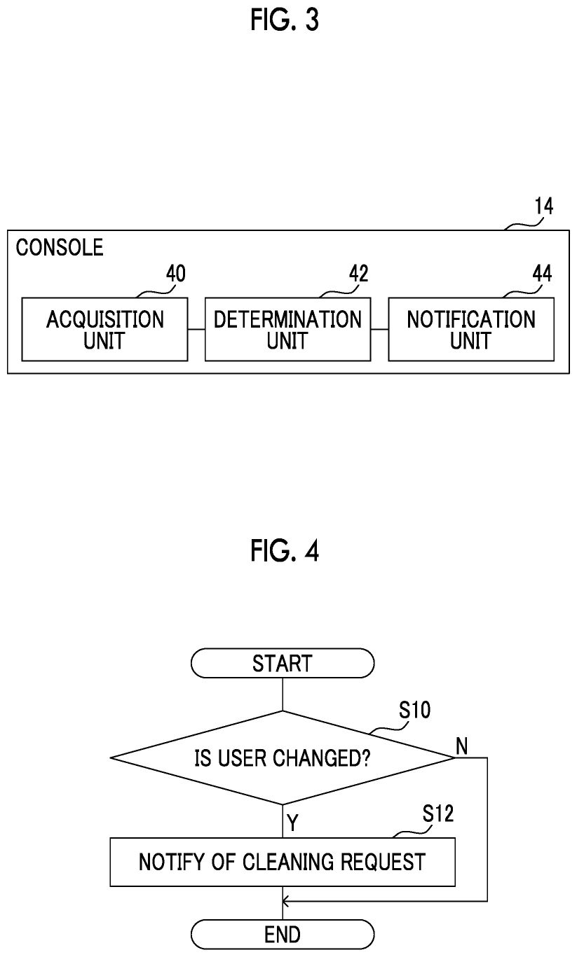

Next, a functional configuration of the console 14 according to the present embodiment will be described with reference to FIG. 3. As shown in FIG. 3, the console 14 includes an acquisition unit 40, a determination unit 42, and a notification unit 44. The CPU 20 of the console 14 executes the information processing program 30 to function as the acquisition unit 40, the determination unit 42, and the notification unit 44.

The acquisition unit 40 acquires an imaging order transmitted from a management system that manages examinations in the hospital, such as a radiology information system (RIS). The imaging order includes, for example, identification information of a subject imaged by the imaging device 12, such as an imaging region, radiation irradiation conditions, and a patient name, and identification information of a user who operates a mobile information terminal 16 such as a radiologist. In addition, the acquisition unit 40 acquires, as status information of the imaging device 12, imaging end information indicating that the imaging of the subject has been ended which is transmitted from the imaging device 12. The imaging end information may be transmitted from the mobile information terminal 16 as the status information of the mobile information terminal 16.

In a case in which the imaging end information is acquired by the acquisition unit 40, the determination unit 42 determines whether or not the user is changed by using the imaging order. In the present embodiment, the next imaging order is transmitted to the console 14 before the imaging based on one imaging order by the imaging device 12 is ended and the user is changed. In other words, the next imaging order is transmitted to the console 14 before the users are switched and the next user is in contact with the mobile information terminal 16. That is, the imaging order is information that can be acquired by the console 14 even in a state in which the next user is not in contact with the mobile information terminal 16 before the user is changed. In addition, it can be said that the imaging order is information that can be acquired by the console 14 even in a state in which the next user is not in contact with the mobile information terminal 16 before the subject is changed.

The notification unit 44 notifies of the cleaning request of the mobile information terminal 16 in a case in which the determination unit 42 determines that the user is to be changed. Specifically, the notification unit 44 transmits a message indicating a cleaning request of the mobile information terminal 16 to the mobile information terminal 16. The mobile information terminal 16 notifies of the cleaning request by displaying the message transmitted from the notification unit 44 on a display of the mobile information terminal 16. Accordingly, the user who operates the mobile information terminal 16 can recognize that it is the timing to clean the mobile information terminal 16 before contacting the mobile information terminal 16, based on the next imaging order. Therefore, it is possible to suppress the spread of the infectious disease via the mobile information terminal 16.

The user cleans the mobile information terminal 16 by, for example, irradiating an outer surface of the mobile information terminal 16 with ultra violet (UV) having a wavelength of 222 nm. In a case in which the console 14 is shared, the notification unit 44 may also notify of the cleaning request of the console 14. In addition, a cleaning method of the mobile information terminal 16 is not limited to UV irradiation, and disinfection with an alcohol disinfectant solution may be used, or a plurality of cleaning methods may be combined.

Next, an action of the console 14 according to the present embodiment will be described with reference to FIG. 4. The cleaning request notification processing shown in FIG. 4 is executed by executing the information processing program 30 by the CPU 20 of the console 14. The cleaning request notification processing shown in FIG. 4 is executed, for example, in a case in which the acquisition unit 40 of the console 14 acquires the imaging end information transmitted from the imaging device 12. In addition, it is assumed that the acquisition unit 40 has acquired the imaging order for the next imaging at the timing when the acquisition unit 40 acquires the imaging end information.

In step S10 of FIG. 4, the determination unit 42 determines whether or not the user is changed by determining whether or not the identification information of the user included in the immediately preceding imaging order and the identification information of the user included in the next imaging order are different from each other. In a case in which this determination results in a positive determination, the processing proceeds to step S12.

In step S12, the notification unit 44 notifies of the cleaning request of the mobile information terminal 16 as described above. When the processing in step S12 is ended, the cleaning request notification processing is ended. In addition, in a case in which the determination in step S10 results in a negative determination, the cleaning request notification processing is ended.

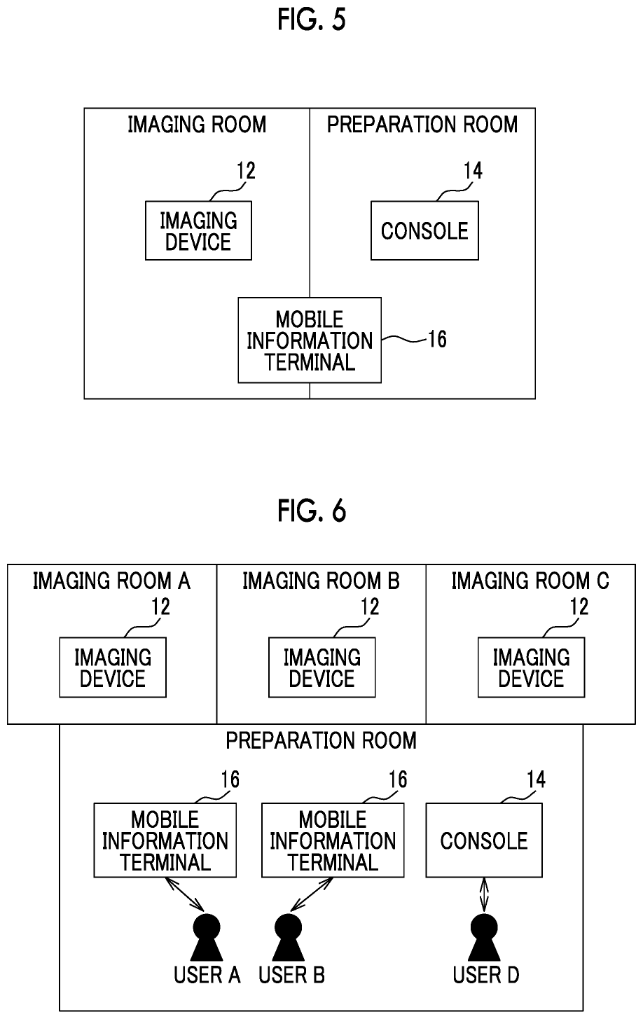

Next, specific examples will be described. As shown in FIG. 5, the imaging device 12 is installed in an imaging room, the console 14 is installed in a preparation room, and the mobile information terminal 16 is configured to be portable between the imaging room and the preparation room.

A user A guides a subject A to the imaging room, and performs confirmation of the subject A and confirmation of imaging conditions while observing a first imaging order displayed on the display of the mobile information terminal 16. In addition, the user A performs preparation for imaging such as positioning of the subject A, and moves to the preparation room after the preparation for imaging is completed. In addition, the user A starts imaging with the imaging device 12 by performing an imaging start operation such as pressing a radiation irradiation button in the preparation room. After the imaging is completed, the user A guides the subject A to the outside of the imaging room.

Next, the user A guides a subject B to the imaging room, and performs confirmation of the subject B and confirmation of imaging conditions while observing a second imaging order displayed on the display of the mobile information terminal 16. At this time, since the identification information of the user included in the first imaging order and the identification information of the user included in the second imaging order are the same, the cleaning request of the mobile information terminal 16 is not notified. The user A, similar to the subject A, performs imaging of the subject B with the imaging device 12, and then guides the subject B to the outside of the imaging room.

Next, a user B guides a subject C to the imaging room. Since identification information of the user included in a third imaging order related to imaging of the subject C and the identification information of the user included in the second imaging order are different from each other, after the imaging based on the second imaging order is completed, the cleaning request of the mobile information terminal 16 is notified. Therefore, the user B can recognize that it is the timing to clean the mobile information terminal 16 before contacting the mobile information terminal 16. Therefore, the user B performs the imaging of the subject C with the imaging device 12 after cleaning the mobile information terminal 16.

Next, as shown in FIG. 6, an example in which three imaging devices 12 are installed one by one in three imaging rooms, one preparation room is prepared for the three imaging rooms, and one console 14 is installed in one preparation room will be described as an example. In the example in FIG. 6, two mobile information terminals 16 are prepared, two users A and B are assigned to the two mobile information terminals 16, and one user D is assigned to the console 14.

In such a situation, as shown in FIG. 6, it is preferable that one of the two mobile information terminals 16 is fixedly assigned to the user A, and the other of the two mobile information terminals 16 is fixedly assigned to the user B. As a result, it is possible to reduce frequency of notification of the cleaning request.

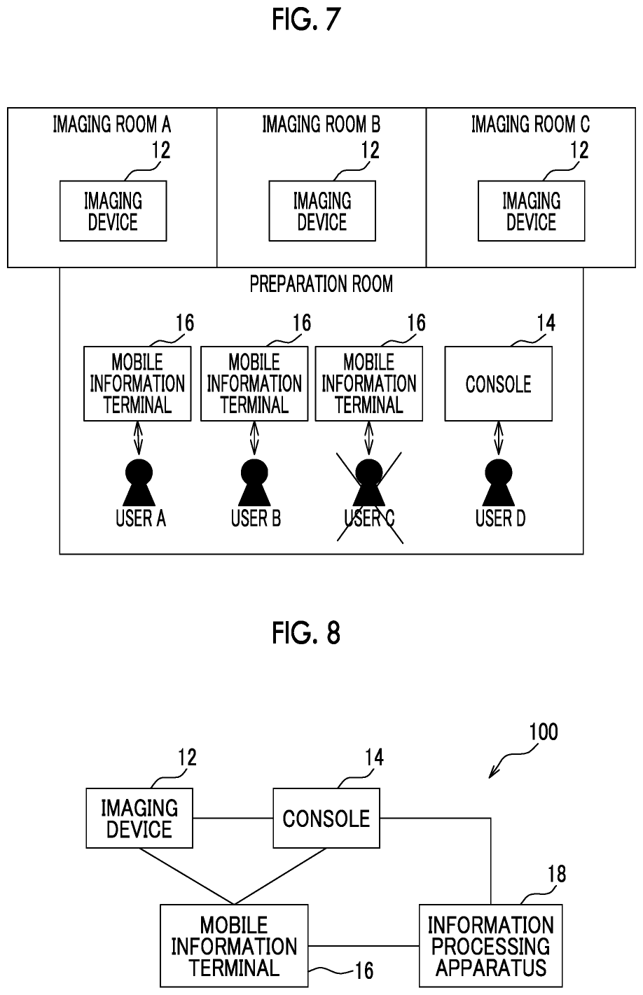

Next, an example in which the number of mobile information terminals 16 is larger than the number of users. As an example, as shown in FIG. 7, this occurs in a situation where three users A, B, and C are assigned to the three mobile information terminals 16 one by one, and one user (user C in the example of FIG. 7) is absent. In this case, by stopping the use of the mobile information terminal 16 assigned to the user C, the frequency of notification of the cleaning request can be reduced. From the above, from the viewpoint of reducing the frequency of notification of the cleaning request, it is preferable to assign the mobile information terminal 16 to the user rather than the imaging room.

In addition, in order to reduce the frequency of notification of the cleaning request, it is preferable that the user does not bring the mobile information terminal 16 into the preparation room and uses the mobile information terminal 16 only in the imaging room. In addition, in a case in which there are two or more users, it is preferable to separate them into a user dedicated to the imaging room and a user dedicated to the preparation room. In this case, it is preferable that the user dedicated to the imaging room wear protective clothing.

As described above, according to the present embodiment, since the notification of the cleaning request of the object is appropriately performed, it is possible to suppress the spread of the infectious disease through the object.

In the first embodiment, the mobile information terminal 16 may have the functions of the acquisition unit 40, the determination unit 42, and the notification unit 44. In this case, the mobile information terminal 16 also serves as an information processing apparatus according to the disclosed technology and a control device that controls the imaging device 12.

In this form example, the mobile information terminal 16 may determine whether or not the user is changed by using the identification information of the user obtained by the short-range wireless communication. In this case, for example, the mobile information terminal 16 acquires the identification information of the user from an identification (ID) card possessed by the user and embedded with an integrated circuit (IC) chip in which the identification information of the user is recorded.

In addition, in the first embodiment, the example of notifying the cleaning request in a case in which the user is changed has been described, but the present disclosure is not limited thereto. For example, a form of notifying of the cleaning request in a case in which the subject is changed may be used. In addition, in this form example, in a case in which the notification unit 44 detects that the imaging of the subject with the imaging device 12 has been ended, the notification unit 44 may notify the cleaning request by considering that the subject is changed. For example, the notification unit 44 detects that the imaging of the subject with the imaging device 12 has been ended in a case in which the acquisition unit 40 acquires the imaging end information transmitted from the imaging device 12. In addition, for example, the notification unit 44 may detect that the imaging of the subject with the imaging device 12 has been ended in a case in which information indicating that the imaging has been ended is input by the user.

In addition, for example, the mobile information terminal 16 may determine whether or not the user is changed by using the image information of the user imaged by the camera installed in the mobile information terminal 16, who approaches the mobile information terminal 16 within a certain distance.

In addition, in the first embodiment, the notification unit 44 may further notify the cleaning request of the imaging device 12 paired with the mobile information terminal 16.

In addition, in the first embodiment, the notification unit 44 may notify a warning in a case in which cleaning of the mobile information terminal 16 has not been performed even after a certain period of time has elapsed after the notification of the cleaning request of the mobile information terminal 16. Whether or not the cleaning of the mobile information terminal 16 has been performed can be determined by a cleaner inputting information indicating that the cleaning has been ended after cleaning of the mobile information terminal 16.

In addition, in the first embodiment, the notification unit 44 may notify of a warning in a case in which a period of time from the notification of the cleaning request of the mobile information terminal 16 to a next use of the mobile information terminal 16 for which cleaning has not been performed is equal to or less than a predetermined threshold value. The threshold value in this case is set in advance according to a policy of the hospital and the like. This warning is erased in a case in which the mobile information terminal 16 is cleaned. Examples of warning notification in this case include displaying a warning message on the display of the mobile information terminal 16, lighting or turning a light emitting diode (LED) lamp of the mobile information terminal 16 on and off, and sounding a warning sound from a speaker of the mobile information terminal 16. In addition, examples of warning notification in this case include transmission of a warning message to a mobile information terminal possessed by a person in charge predetermined as an administrator of the mobile information terminal 16 or an e-mail address of the person in charge.

In addition, the control to prohibit use of the mobile information terminal 16 which is a target to be notified of the warning may be further performed until the cleaning of the mobile information terminal 16 is performed. The control for prohibiting the use of the mobile information terminal 16 includes, for example, canceling the pairing with the imaging device 12.

In addition, in the first embodiment, in a case in which the imaging order does not include the identification information of the user, and in a case in which the user is changed, delimiter information indicating that the user is changed may be input between the imaging order and the imaging order. In this case, it is determined whether or not the user is changed depending on whether or not there is delimiter information between two consecutive imaging orders.

In addition, in the first embodiment, in a case in which the mobile information terminal 16 is not operated for a period of time set in advance (for example, 5 minutes) or longer, the cleaning request may be notified by considering that the operation by the user is completed and then the operation is to be performed by a different user.

### Second Embodiment

In the first embodiment, an example of notifying of the cleaning request based on the imaging order has been described. In the second embodiment, an example of notifying of the cleaning request based on information other than the imaging order will be described.

First, a configuration of an information processing system 100 according to the present embodiment will be described with reference to FIG. 8. The same components as components in the first embodiment are denoted by the same reference numerals, and a description thereof will be omitted. As shown in FIG. 8, the information processing system 100 includes an imaging device 12, a console 14, a mobile information terminal 16, and an information processing apparatus 18. The information processing apparatus 18 is communicably connected to the console 14 and the mobile information terminal 16.

Since the hardware configuration of the information processing apparatus 18 is the same as that of the console 14 (see FIG. 2), description thereof will be omitted.

In the present embodiment, entry/exit information of the user with respect to the imaging room in which the mobile information terminal 16 is installed is accumulated in the information processing apparatus 18 by short-range wireless communication such as an IC tag. Specifically, an IC tag reader is installed in the vicinity of a doorway of the imaging room. Then, the user holds the ID card in which the IC chip in which the identification information of the user is recorded is embedded over the IC tag reader in a case of entering the imaging room and in a case of exiting the imaging room. Accordingly, the identification information of the user is read by the IC tag reader, and the read identification information of the user is transmitted to the information processing apparatus 18. The entry/exit information is information that can be acquired by the information processing apparatus 18 even in a state in which the next user is not in contact with the mobile information terminal 16 before the user is changed.

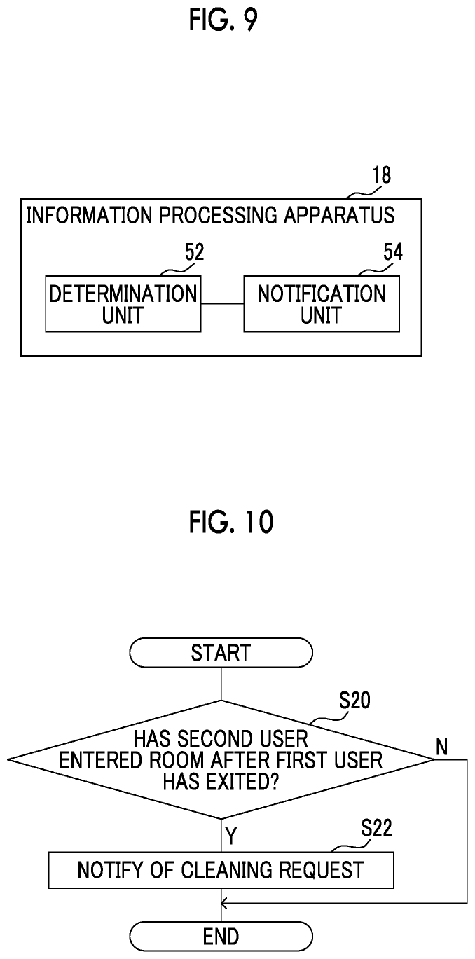

Next, a functional configuration of the information processing apparatus 18 according to the present embodiment will be described with reference to FIG. 9. As shown in FIG. 9, the information processing apparatus 18 includes a determination unit 52 and a notification unit 54. The CPU 20 of the information processing apparatus 18 executes the information processing program 30 to function as the determination unit 52 and the notification unit 54.

The determination unit 52 determines whether or not a second user different from a first user has entered the imaging room in which the mobile information terminal 16 is installed after the first user has exited the imaging room by using entry/exit information of the user with respect to the imaging room.

Similar to the notification unit 44, the notification unit 54 notifies of the cleaning request of the mobile information terminal 16 in a case in which the determination unit 52 determines that the second user different from the first user has entered the imaging room after the first user has exited the imaging room.

Next, the action of the information processing apparatus 18 according to the present embodiment will be described with reference to FIG. 10. The cleaning request notification processing shown in FIG. 10 is executed by executing the information processing program 30 by the CPU 20 of the information processing apparatus 18. The cleaning request notification processing shown in FIG. 10 is executed, for example, in a case in which the information processing apparatus 18 receives the entry/exit information into the imaging room.

In step S20 in FIG. 10, the determination unit 52 determines whether or not a second user different from a first user has entered the imaging room in which the mobile information terminal 16 is installed after the first user has exited the imaging room by using entry/exit information of the user with respect to the imaging room. In a case in which this determination results in a positive determination, the processing proceeds to step S22.

In step S22, the notification unit 54 notifies of the cleaning request of the mobile information terminal 16 as described above. When the processing in step S22 is ended, the cleaning request notification processing is ended. In addition, in a case in which the determination in step S20 results in a negative determination, the cleaning request notification processing is ended.

As described above, according to the present embodiment, the same effect as the first embodiment can be accomplished.

In the second embodiment, the determination unit 52 may use a motion picture captured at a predetermined frame rate by a camera that images the vicinity of the doorway of the imaging room as the entry/exit information of the user with respect to the imaging room in which the mobile information terminal 16 is installed. In this case, the determination unit 52 determines whether or not the second user different from the first user has entered the imaging room after the first user has exited the imaging room by performing image analysis processing on the motion picture captured by the camera.

In addition, the following various processors can be used in each of the above-described embodiments as the hardware structures of processing units, which perform various types of processing, such as the acquisition unit 40, the determination units 42 and 52, and the notification units 44 and 54. The various processors include, in addition to a CPU that is a general-purpose processor functioning as various processing units by executing software (program) as described above, a programmable logic device (PLD) that is a processor such as a field programmable gate array (FPGA) having a circuit configuration changeable after manufacturing, a dedicated electric circuit that is a processor such as an application specific integrated circuit (ASIC) having a circuit configuration dedicatedly designed to execute specific processing, and the like.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

As an example of configuring the plurality of processing units with one processor, first, as represented by a computer such as a client and a server, a form of configuring one processor with a combination of one or more CPUs and software and causing the processor to function as the plurality of processing units is present. As a second example, as represented by a system on chip (SoC) or the like, there is a form in which a processor that realizes the function of the entire system including the plurality of processing units by one integrated circuit (IC) chip is used. Accordingly, the various processing units are configured using one or more of the various processors as a hardware structure.

Furthermore, more specifically, an electric circuit (circuitry) in which circuit elements such as semiconductor elements are combined can be used as the hardware structure of the various processors.

In addition, an aspect in which the information processing program 30 is stored (installed) in the storage unit 22 in advance has been described in each of the above-described embodiments, but the present disclosure is not limited thereto. The information processing program 30 may be provided in a form in which the information processing program 30 is recorded in recording mediums, such as a compact disc read only memory (CD-ROM), a digital versatile disc read only memory (DVD-ROM), and a universal serial bus (USB) memory. In addition, the information processing program 30 may be downloaded from an external device through a network.

The entire content of the present disclosure of Japanese Patent Application No. 2020-180853, filed Oct. 28, 2020, is incorporated in the present specification by reference. All documents, patent applications, and technical standards disclosed in the present specification are incorporated in the present specification by reference to the same extent as in a case where each of the documents, patent applications, technical standards are specifically and individually indicated to be incorporated by reference.

What is claimed is:

1. An information processing apparatus comprising:
at least one processor,
wherein the processor is configured to, before at least one of a user who operates a control device controlling an imaging device that captures a medical image or a subject to be imaged by the imaging device is changed, notify of a cleaning request of the control device based on information acquirable in a noncontact manner with the control device.

2. The information processing apparatus according to claim 1,
wherein the information is an imaging order including identification information of the user, and
the processor is configured to:
determine whether or not the user is changed by using the imaging order; and
notify of the cleaning request in a case in which it is determined that the user is changed.

3. The information processing apparatus according to claim 1,
wherein the processor is configured to:
determine whether or not the user is changed by using identification information of the user obtained by short-range wireless communication with the control device; and
notify of the cleaning request in a case in which it is determined that the user is changed.

4. The information processing apparatus according to claim 1,
wherein the processor is configured to notify of the cleaning request in a case in which it is detected that a second user different from a first user has entered an imaging room in which the control device is installed after the first user has exited the imaging room by using entry/exit information of the user with respect to the imaging room.

5. The information processing apparatus according to claim 1,
wherein the processor is configured to:
determine whether or not the user is changed by using image information of the user imaged by a camera, who approaches the control device within a certain distance; and
notify of the cleaning request in a case in which it is determined that the user is changed.

6. The information processing apparatus according to claim 1,
wherein the processor is configured to notify of the cleaning request by considering that the subject is changed in a case in which it is detected that imaging of the subject with the imaging device has been ended.

7. The information processing apparatus according to claim 6,
wherein the processor is configured to detect that the imaging of the subject has been ended by using at least one of status information of the imaging device or status information of the control device.

8. The information processing apparatus according to claim 1,
wherein the processor is configured to further notify of the cleaning request of the imaging device paired with the control device.

9. The information processing apparatus according to claim 1,
wherein the processor is configured to notify of a warning in a case in which cleaning of the control device has not been performed even after a certain period of time has elapsed after the notification of the cleaning request.

10. The information processing apparatus according to claim 1,
wherein the processor is configured to notify of a warning in a case in which a period of time from the notification of the cleaning request to a next use of the control device for which cleaning has not been performed is equal to or less than a predetermined threshold value.

11. The information processing apparatus according to claim 9,
wherein the processor is configured to further perform control to prohibit use of the control device which is a target to be notified of the warning.

12. An information processing method executed by a processor included in an information processing apparatus, the method comprising:
notifying, before at least one of a user who operates a control device controlling an imaging device that captures a medical image or a subject to be imaged by the imaging device is changed, of a cleaning request of the control device based on acquirable information in a noncontact manner with the control device.

13. A non-transitory computer-readable storage medium storing an information processing program for causing a processor included in an information processing apparatus to execute a process comprising:
notifying, before at least one of a user who operates a control device controlling an imaging device that captures a medical image or a subject to be imaged by the imaging device is changed, of a cleaning request of the control device based on acquirable information in a noncontact manner with the control device.
